(19) United States
(12) Patent Application Publication
(10) Pub. No.: US 2014/0214002 A1
(43) Pub. Date: Jul. 31, 2014

(54) BIFURCATION CATHETER WITH VARIABLE LENGTH OCCLUSION ELEMENTS

(71) Applicant: THERMOPEUTIX, INC., San Diego, CA (US)

(72) Inventors: GLEN LIEBER, POWAY, CA (US); RONALD JAY SOLAR, SAN DIEGO, CA (US); JOHN GILBERT, BROOKLINE, MA (US)

(73) Assignee: THERMOPEUTIX, INC., SAN DIEGO, CA (US)

(21) Appl. No.: 13/751,093

(22) Filed: Jan. 27, 2013

(51) Int. Cl. A61M 25/10

(52) U.S. Cl. CPC A61M 25/1011
USPC 604/509; 604/101.05

(57) ABSTRACT

There is provided a catheter for providing a delivery substance to a bifurcated vessel, and isolating a treatment zone within the bifurcation. The catheter includes a proximal shaft and a first and second distal shaft positioned within the proximal shaft. The proximal shaft has a proximal occlusion element at a distal end thereof, the first distal shaft has a first distal occlusion element at a distal end thereof, and the second distal shaft has a second distal occlusion element at a distal end thereof. When the proximal occlusion element, and the first and second distal occlusion elements are deployed, a treatment zone is defined, and a delivery substance may be introduced into the treatment zone via the proximal shaft.

### FIELD OF THE INVENTION

The present invention is directed to a catheter and methods of using the catheter for providing a delivery substance to a treatment zone in a bifurcated vessel. More specifically, the present invention is directed to a catheter and methods of using the catheter for providing a delivery substance such as a drug solution to a bifurcated vessel, and isolating an area around the bifurcation for providing the delivery substance.

### BACKGROUND OF THE INVENTION

Methods and devices designed to provide drugs to a vessel, include, for example, the use of drug coated balloons, such as disclosed in U.S. Pat. No. 5,954,706 to Sahatjian, for example. Such devices include a catheter with an expandable portion, wherein at least a portion of the exterior surface of the expandable portion is defined by a coating of hydrogel polymer. Incorporated within the hydrogel polymer is a solution of a preselected drug to be delivered to the tissue or plaque. Disadvantages of such devices include the need to choose a particular drug and dosage in advance, as well as limitations on the length and diameter of the treatment area as defined by the predetermined length and diameter of the expandable portion, since these devices often work by direct contact of the device to the vessel.

Another device is disclosed in U.S. Pat. No. 6,287,320 to Slepian. A catheter includes first and second expansile members which are expanded to occlude a diseased region, and a therapeutic agent is introduced into the diseased region via the catheter. The catheter is allowed to remain in place for a therapeutically effective amount of time to allow the therapeutic agent to contact the diseased portion for such a period of time.

Another device is disclosed in US Patent Publication 2007/0078433 to Schwager et al. This device includes a balloon catheter having a predetermined inflow angle of medication. A first and second balloon are positioned on the catheter, with a treatment zone therebetween. Disadvantages of devices such as the ones disclosed in the above-referenced publications include limitations on the length of the treatment area as predetermined by the distance between the expansile members.

A device disclosed in US Patent Application Publication Number 2005/0059930 to Garrison et al. includes a catheter system with at least two expandable occluding elements which are used to create a localized site for administration of agents. The catheters are slidable with respect to one another to vary the space between the balloons as desired. However, the localized site is prone to overpressure since there is no disclosed way to remove excess fluid from the site.

WO Patent Publication Number WO/2012/137177 to Solar et al. discloses a catheter system having an inner elongated element, an outer elongated element coaxial to the inner elongated element, a proximal occlusion element positioned at the distal end of the outer elongated element, proximal to an outlet port and a distal occlusion element positioned at a distal end of the inner elongated element. The distal end of the inner elongated element is distal to and movable with respect to the outer elongated element distal end. This provides for a variable length catheter system which can define a treatment zone. However, Solar et al. do not disclose a catheter system which would be useful in treating a bifurcated vessel.

There is thus a need for a catheter system and method which can provide a solution for isolating a treatment zone in a bifurcated vessel and providing a treatment solution or other substance to the treatment zone.

### SUMMARY OF THE INVENTION

There is provided, in accordance with embodiments of the present invention, a catheter for delivery of agents to a bifurcated vessel. The catheter includes a proximal shaft having a proximal shaft proximal end, distal end and outer wall extending from the proximal shaft proximal end to the proximal shaft distal end, wherein the outer wall defines a proximal shaft lumen, a proximal occlusion element positioned at the proximal shaft distal end, a first distal shaft positioned within the proximal shaft lumen and having a first distal shaft proximal end, distal end, and outer wall extending from the first distal shaft proximal end to the first distal shaft distal end, a first distal occlusion element positioned at the first distal shaft distal end, a second distal shaft positioned within the proximal shaft lumen and having a second distal shaft proximal end, distal end, and outer wall extending from the second distal shaft proximal end to the second distal shaft distal end, and a second distal occlusion element positioned at the second distal shaft distal end.

In accordance with further features in embodiments of the invention, the catheter may further include a first distal rail at the first distal shaft distal end, a second distal rail at the second distal shaft distal end. In accordance with further features in embodiments of the invention, the catheter may include a first distal shaft lumen, and a second distal shaft lumen. In some embodiments, the first distal shaft is slidingly movable with respect to the proximal shaft and the first distal occlusion element is at a variable distance from the proximal occlusion element. In some embodiments, the second distal shaft is slidingly movable with respect to the proximal shaft and the second distal occlusion element is at a variable distance from the proximal occlusion element. In other embodiments, the first and/or second distal shaft is attached to the proximal shaft, and the first and/or second distal occlusion elements are at a fixed distance from the proximal occlusion element. In accordance with further features in embodiments of the invention, the catheter may further include radiopaque markers, including a marker at the proximal occlusion element distal end, and at the first and second distal occlusion element proximal ends.

There is provided, in accordance with embodiments of the present invention, a method of treating a bifurcated vessel. The method includes providing a catheter having a proximal shaft with a proximal occlusion element at a distal end thereof, a first distal shaft positioned within the proximal shaft having a first distal occlusion element at a distal end thereof, and a second distal shaft positioned within the proximal shaft having a second distal occlusion element at a distal end thereof, placing a first movable guidewire into a first branch vessel, placing a second movable guidewire into a second branch vessel, positioning the first distal shaft on the first movable guidewire and the second distal shaft on the second movable guidewire, advancing the catheter into the bifurcated vessel via the first and second movable guidewires, positioning the proximal shaft in a proximal portion of the bifurcated vessel, positioning the first distal shaft in a first distal branch portion of the bifurcated vessel, positioning the second distal shaft in a second distal branch portion of the bifurcated vessel, after the positioning of the proximal shaft, first distal shaft and second distal shaft, deploying the proximal occlusion element, and the first and second distal occlusion elements, and introducing a delivery substance through the proximal shaft into the vessel.

In accordance with further features in embodiments of the invention, the method may include advancing the catheter by advancing the proximal shaft, and first and second distal shafts simultaneously, or by advancing the first and second distal shafts first, and then advancing the proximal shaft over the first and second distal shafts. In accordance with further features in embodiments of the present invention, advancing the catheter may be done by placing the first movable guidewire through a first distal rail positioned on a distal end of the first distal shaft, and/or placing the second movable guidewire through a second distal rail positioned on a distal end of the second distal shaft. In accordance with additional embodiments, the advancing may be done by placing the first movable guidewire through a first distal shaft lumen, and/or placing the second movable guidewire through a second distal shaft lumen. The method may further include, after a period of time following the introducing of a delivery substance, removing the delivery substance from the vessel through the proximal shaft, undeploying the proximal occlusion element, the first distal occlusion element, and the second distal occlusion element, and removing the catheter from the vessel.

Unless otherwise defined, all technical and scientific terms used herein have the same meaning as commonly understood by one of ordinary skill in the art to which this invention belongs. Although methods and materials similar or equivalent to those described herein can be used in the practice or testing of the embodiments of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of various embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several embodiments of the invention may be embodied in practice.

In the drawings:

FIG. 1A is a perspective illustration of a catheter in accordance with embodiments of the present invention;

FIG. 1B is a cross-sectional illustration of the catheter of FIG. 1A;

FIGS. 2A-2C are perspective illustrations showing a close-up view of a proximal shaft distal end of the catheter of FIGS. 1A and 1B, and depicting a proximal shaft inlet/outlet port in accordance with embodiments of the present invention;

FIGS. 3A-3F are schematic illustrations (FIGS. 3A, 3C and 3E) and cross-sectional illustrations (FIGS. 3B, 3D and 3F) showing first and second distal shafts of the catheter of FIGS. 1A and 1B, positioned within the proximal shaft of the catheter of FIGS. 1A and 1B, in accordance with embodiments of the present invention;

FIGS. 4A-4F are schematic illustrations (FIGS. 4A and 4D) and cross-sectional illustrations (FIGS. 4B, 4C, 4E and 4F), respectively, of first distal shaft of the catheter of FIGS. 1A and 1B, with a first distal rail in accordance with embodiments of the present invention;

FIG. 5 is an illustration of the first distal shaft of the catheter of FIGS. 1A and 1B, having a fixed wire balloon, in accordance with yet another embodiment of the present invention;

FIGS. 6A-6G are schematic illustrations showing a method of using the catheter of FIGS. 1A and 1B, in accordance with embodiments of the present invention;

FIGS. 7A-7C are schematic illustrations showing a method of using the catheter of FIGS. 1A and 1B, in accordance with additional embodiments of the present invention;

FIGS. 8A and 8B are schematic illustrations showing a method of using the catheter of FIGS. 1A and 1B, in accordance with yet additional embodiments of the present invention; and

FIGS. 9A-9E are angiographic images taken during a procedure using the catheter of FIGS. 1A and 1B in an experimental porcine model.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF THE INVENTION

In the following detailed description, numerous specific details are set forth in order to provide a thorough understanding of the present invention. It will be understood by those of ordinary skill in the art that embodiments of the present invention may be practiced without these specific details. In other instances, well-known methods, procedures, components and structures may not have been described in detail so as not to obscure the present invention.

The present invention relates to a catheter for providing a treatment solution to a bifurcated vessel. Further advantages of the design of the catheter of the present invention will be described hereinbelow.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

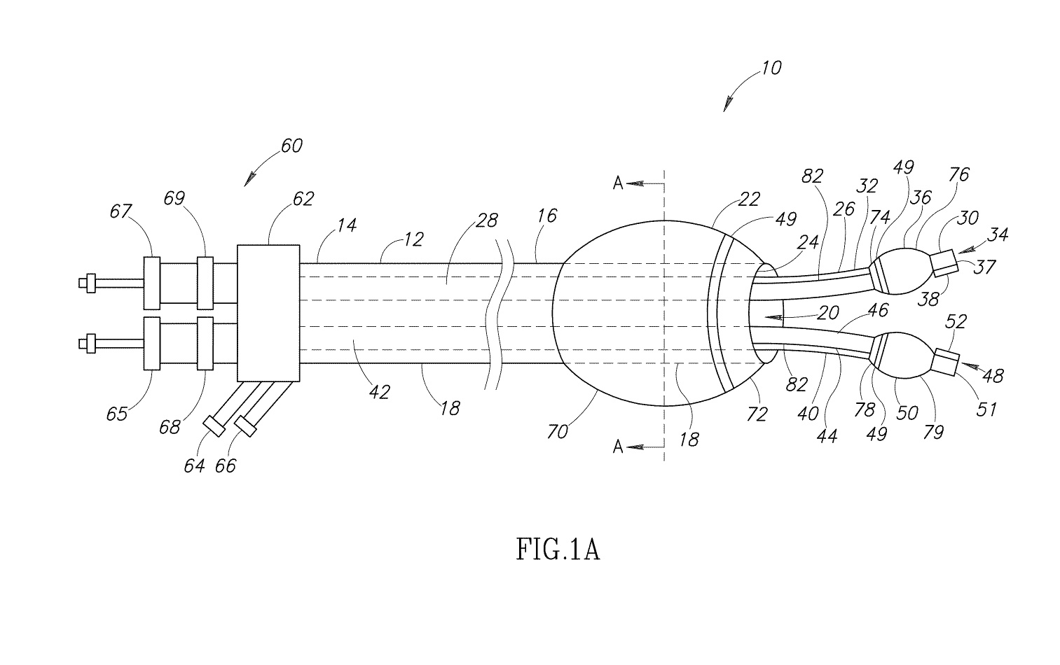

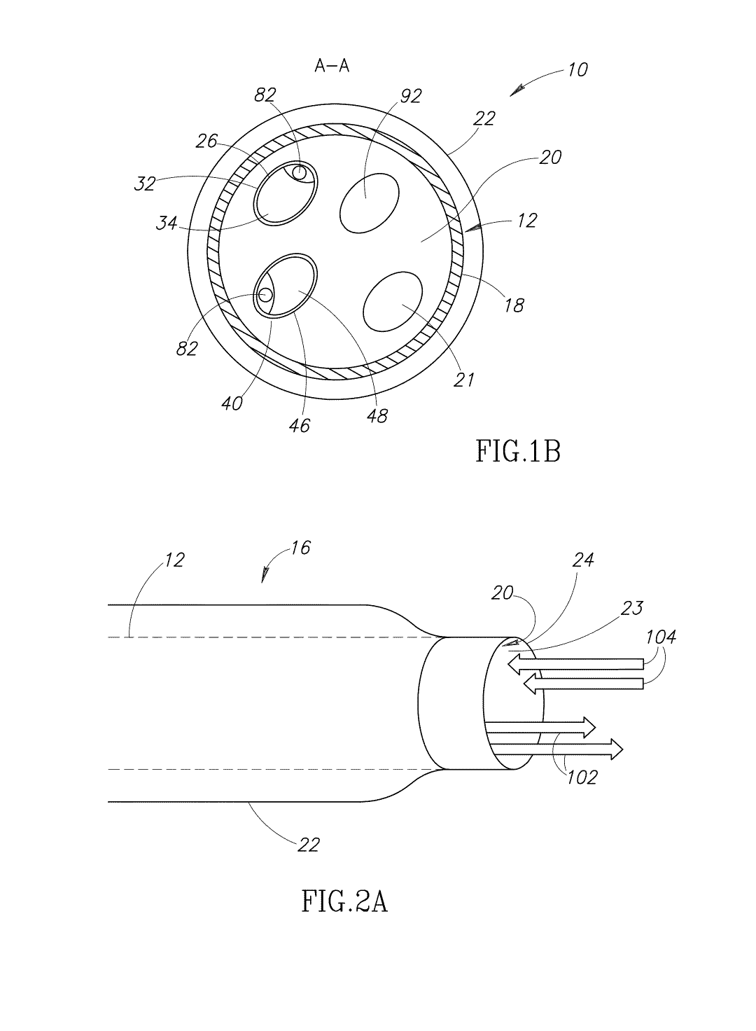

Reference is now made to FIGS. 1A and 1B, which are a perspective and cross-sectional illustration, respectively, of a catheter 10 in accordance with embodiments of the present invention. Catheter 10 includes a proximal shaft 12 having a proximal shaft proximal end 14 and a proximal shaft distal end 16. Proximal shaft 12 is preferably an elongated tubular member, including a proximal shaft outer wall 18 and a proximal shaft lumen 20 internal to proximal shaft outer wall 18. Proximal shaft outer wall 18 may be of any shape suitable for a catheter shaft, such as cylindrical or ovoid, for example and may be comprised of any suitable material, such as a metal or a polymeric material, for example. Proximal shaft proximal end 14 is connected to a hub 60 for introduction of agents, guidewires, fluids, drug solution, contrast, diagnostic solution, or other substances as will be described further hereinbelow. A proximal occlusion element 22 and a proximal shaft inlet/outlet port 24 are positioned at or near proximal shaft distal end 16. Proximal occlusion element 22 is positioned proximal to proximal shaft inlet/outlet port 24.

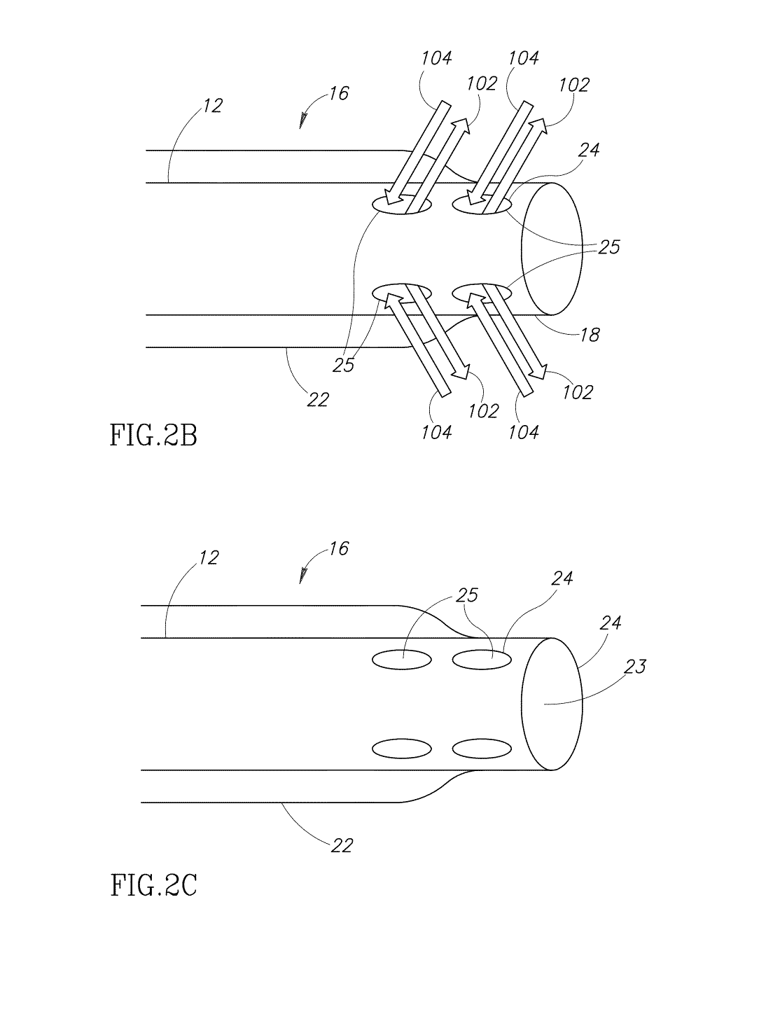

Reference is now made to FIGS. 2A-2C, which are perspective illustrations showing a close-up view of proximal shaft distal end 16 and depicting proximal shaft inlet/outlet port 24 in accordance with embodiments of the present invention. In some embodiments, as shown schematically in FIG. 2A, proximal shaft inlet/outlet port 24 is a proximal shaft distal end opening 23 at proximal shaft distal end 16, wherein proximal shaft distal end opening 23 is continuous with proximal shaft lumen 20, such that any agents introduced through proximal shaft lumen 20 may exit proximal shaft 12 at proximal shaft inlet/outlet port 24 as depicted by outgoing arrows 102, or may enter proximal shaft 12 at proximal shaft inlet/outlet port 24 as depicted by incoming arrows 104. In other embodiments, as shown schematically in FIG. 2B, proximal shaft inlet/outlet port 24 is comprised of one or multiple proximal shaft outer wall openings 25 in proximal shaft outer wall 18, such that any agents introduced through proximal shaft lumen 20 may exit or enter proximal shaft 12 by flowing through proximal shaft outer wall openings 25, as depicted by outgoing arrows 102 and incoming arrows 104, respectively. In other embodiments, as shown in FIG. 2C, proximal shaft inlet/outlet port 24 may include both a proximal shaft distal end opening 23 and one or multiple proximal shaft outer wall openings 25.

Returning now to FIGS. 1A and 1B, catheter 10 further includes at least two distal shafts, including a first distal shaft 26 and a second distal shaft 40. First distal shaft 26 has a first distal shaft proximal end 28 and a first distal shaft distal end 30. First distal shaft 26 is preferably an elongated tubular member, including a first distal shaft outer wall 32. In some embodiments, first distal shaft 26 includes a first distal shaft lumen 34 internal to first distal shaft outer wall 32. First distal shaft outer wall 32 may be of any shape suitable for a catheter shaft, such as cylindrical or ovoid, for example. First distal shaft proximal end 28 is in some embodiments connected to hub 60 for introduction of agents, guidewires, fluids, etc. as will be described further hereinbelow. A first distal occlusion element 36 is positioned at or near first distal shaft distal end 30. In some embodiments, a first distal inlet/outlet port 37 is positioned at first distal shaft distal end 30, and is in fluid communication with first distal shaft lumen 34. Similarly to proximal inlet/outlet port 24, as described with reference to FIGS. 2A-2C, first distal inlet/outlet port 37 may include a distal end opening of first distal shaft 26 or may include multiple outer wall openings on first distal shaft outer wall 32 or both. In some embodiments, first distal occlusion element 36 is positioned proximal to first distal outlet port 37, and first distal outlet port 37 may be used as a guidewire exit port, a perfusion port, or for any other suitable purpose. In other embodiments, first distal occlusion element 36 may be positioned distal to first distal outlet port 37, and first distal outlet port may be used to deliver or remove agents from a treatment zone. In some embodiments, first distal shaft 26 does not include a first distal shaft lumen 34 or a first distal outlet port 37, and a profile of first distal shaft 26 may be reduced.

Second distal shaft 40 has a second distal shaft proximal end 42 and a second distal shaft distal end 44. Second distal shaft 40 is preferably an elongated tubular member, including a second distal shaft outer wall 46. In some embodiments, second distal shaft 40 includes a second distal shaft lumen 48 internal to second distal shaft outer wall 46. Second distal shaft outer wall 46 may be of any shape suitable for a catheter shaft, such as cylindrical or ovoid, for example. Second distal shaft proximal end 42 is in some embodiments connected to hub 60 for introduction of agents, guidewires, fluids, etc. as will be described further hereinbelow. A second distal occlusion element 50 is positioned at or near second distal shaft distal end 44. In some embodiments, a second distal inlet/outlet port 51 is positioned at second distal shaft distal end 44, and is in fluid communication with second distal shaft lumen 48. Similarly to proximal shaft inlet/outlet port 24, as described with reference to FIGS. 2A-2C, second distal inlet/outlet port 51 may include a distal end opening of second distal shaft 40 or may include multiple outer wall openings on second distal shaft outer wall 46 or both. In some embodiments, second distal occlusion element 50 is positioned proximal to second distal outlet port 51, and second distal outlet port 51 may be used as a guidewire exit port, a perfusion port, or for any other suitable purpose. In other embodiments, second distal occlusion element 50 may be positioned distal to second distal outlet port 51, and second distal outlet port 51 may be used to deliver or remove agents from a treatment zone. In some embodiments, second distal shaft 40 does not include a second distal shaft lumen 48 or a second distal outlet port 51, and a profile of second distal shaft 40 may be reduced.

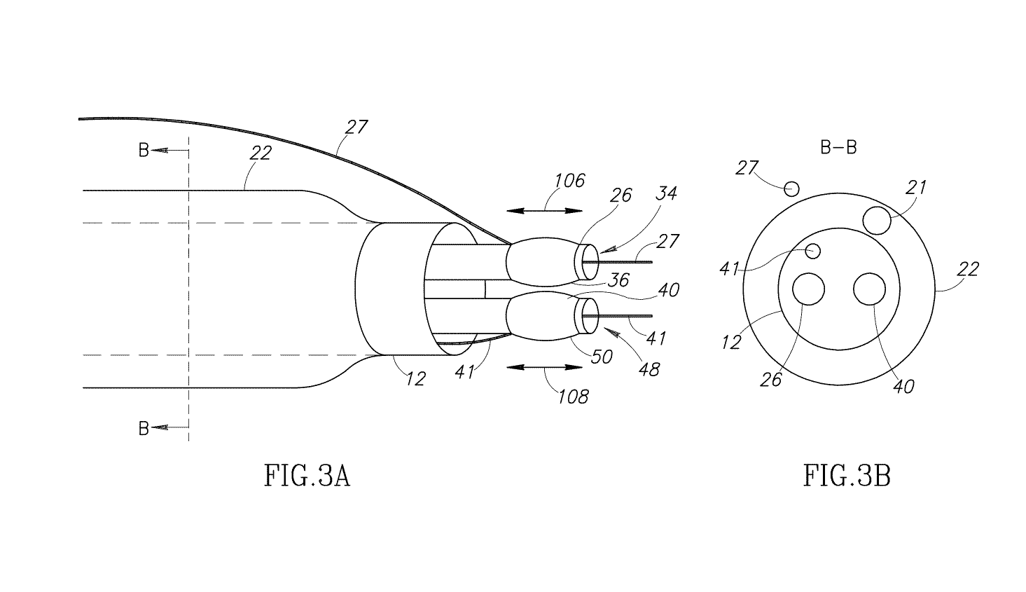

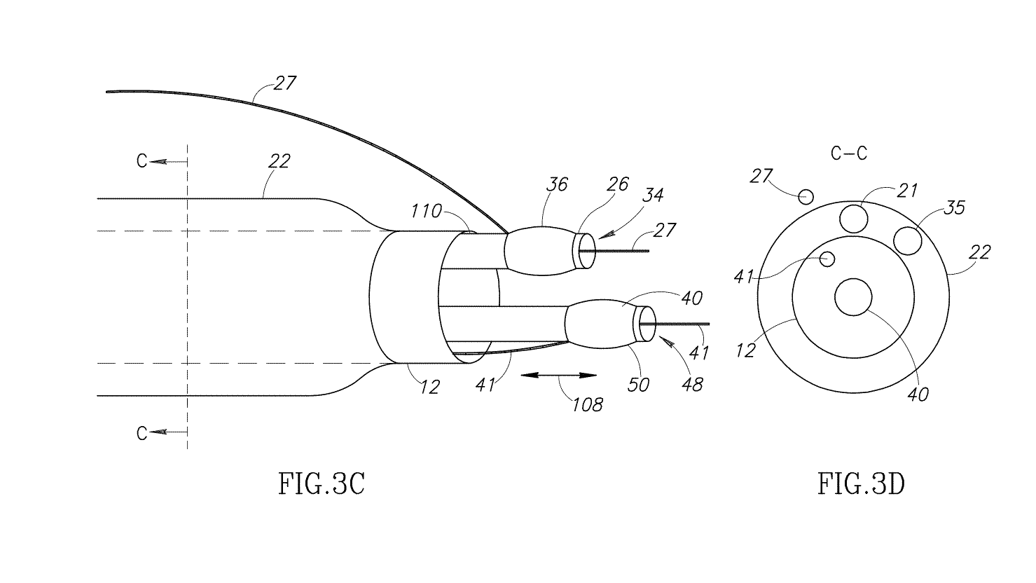

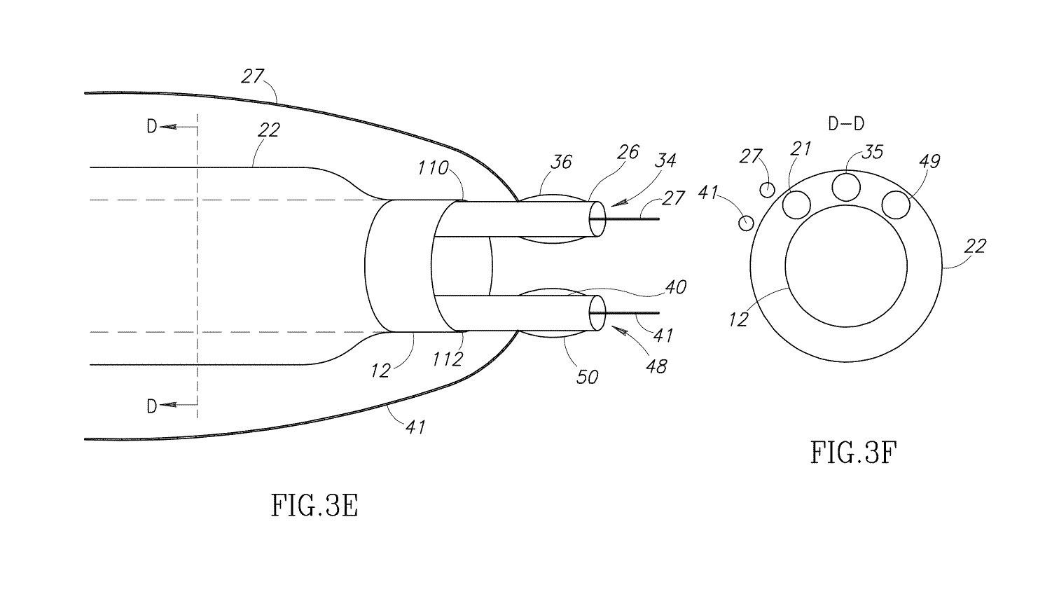

In accordance with embodiments of the present invention, first distal shaft 26 and second distal shaft 40 are both positioned within proximal shaft 12. Reference is now made to FIGS. 3A-3F, which are schematic and cross-sectional illustrations showing embodiments of first and second distal shafts 26 and 40 positioned within proximal shaft 12. In one embodiment, as shown in FIG. 3A schematically and FIG. 3B in cross-section, first distal shaft 26 is slidably movable with respect to proximal shaft 12, as depicted by a first double-sided arrow 106, and second distal shaft 40 is slidably movable with respect to proximal shaft 12, as depicted by a second double sided arrow 108. As such, first distal shaft 26 and second distal shaft 40 may be positioned at variable distances with respect to proximal shaft 12. This allows for use of catheter 10 with different anatomies and for different lengths of a treatment area. As depicted in FIG. 3A, first distal shaft 26 with first distal occlusion element 36 and second distal shaft 40 with second distal occlusion element 50 are positioned within proximal shaft 12. Proximal occlusion element 22 is positioned on proximal shaft 12 with a proximal occlusion element inflation lumen 21 between proximal occlusion element 22 and proximal shaft 12, as depicted in FIG. 3B. In one embodiment, a first distal guidewire 27 is positioned within first distal shaft lumen 34 and exits proximal to first distal occlusion element 36. First distal guidewire 27 then extends proximally outside of proximal shaft 12. In another embodiment, shown schematically with respect to second distal shaft 40, a second distal guidewire 41 is positioned within second distal shaft lumen 48 and exits proximal to second distal occlusion element 50. Second distal guidewire 41 then extends proximally inside of proximal shaft 12. It should be readily apparent that first distal guidewire 27 and second distal guidewire 41 may both extend outside of proximal shaft 12, as shown in FIGS. 3A and 3B with respect to first distal guidewire 27, or first distal guidewire 27 and second distal guidewire 41 may both extend proximally inside of proximal shaft 12, as shown in FIGS. 3A and 3B with respect to second distal guidewire 41. Any combination of these is possible and is included within the scope of the invention. In these embodiments, the need for multiple lumens within proximal shaft 12 results in an outer diameter of proximal shaft 12 of approximately 10 French. An advantage of the embodiment shown in FIGS. 3A and 3B is that each of first and second distal shafts 26 and 40 is configured to be positioned independently and to move with respect to proximal shaft 12, which provides flexibility in the length of the treatment area in each of the branches of the bifurcated vessel.

In another embodiment, as shown in FIG. 3C schematically and in FIG. 3D in cross-section, first distal shaft 26 with first distal occlusion element 36 is fixedly attached to proximal shaft 12, as shown schematically by a first attachment point 110, and second distal shaft 40 is slidably movable with respect to proximal shaft 12, as depicted by second double sided arrow 108. Attachment may be done by adhesive bonding, friction bonding, heat bonding or any other suitable means of attachment. In one embodiment, first distal shaft 26 is built as a continuation of proximal shaft 12. It should be readily apparent that first attachment point 110 may be at any or multiple locations along catheter 10, including at hub 60, along proximal shaft 12, at proximal shaft distal end 16, or at any other suitable location. As depicted in FIG. 3C, second distal shaft 40 with second distal occlusion element 50 is positioned within proximal shaft 12, while first distal shaft 26 with first distal occlusion element 36 is an extension of proximal shaft 12. Proximal occlusion element 22 is positioned on proximal shaft 12 with a proximal occlusion element inflation lumen 21 between proximal occlusion element 22 and proximal shaft 12, as shown in FIG. 3D. A first distal inflation lumen 35 for inflation of first distal occlusion element 36 is also positioned between proximal occlusion element 22 and proximal shaft 12. It should be noted that first distal shaft 26 is not seen as a separate element in FIG. 3D, since at this cross-section first distal shaft 26 is part of proximal shaft 12. First distal guidewire 27 is positioned within first distal shaft lumen 34 and exits proximal to first distal occlusion element 36. First distal guidewire 27 then extends proximally outside of proximal shaft 12. Second distal guidewire 41 is positioned within second distal shaft lumen 48 and exits proximal to second distal occlusion element 50. Second distal guidewire 41 may then extend proximally inside of proximal shaft 12, as shown in FIGS. 3C and 3D, or may extend proximally outside of proximal shaft 12. In these embodiments, the need for multiple lumens within proximal shaft 12 is reduced, since first distal shaft 26 is an extension of proximal shaft 12. This results in an overall reduced profile, ie, in a range of 7 French outer diameter. An advantage over the embodiment shown in FIGS. 3A and 3B is that some flexibility is maintained, while the overall profile is reduced.

In yet another embodiment, as depicted in FIGS. 3E schematically and 3F in cross-section, both first distal shaft 26 with first distal occlusion element 36 and second distal shaft 40 with second distal occlusion element 50 are fixedly attached to proximal shaft 12 at first and second attachment points 110 and 112. Attachment may be done by adhesive bonding, friction bonding, heat bonding or any other suitable means of attachment. In one embodiment, first distal shaft 26 and second distal shaft 40 are built as a continuation of proximal shaft 12. In this embodiment, as depicted in cross-section D-D in FIG. 3F, first distal shaft 26 and second distal shaft 40 are shown as partitioned sections of proximal shaft 12. Proximal occlusion element 22 is positioned on proximal shaft 12 with a proximal occlusion element inflation lumen 21 between proximal occlusion element 22 and proximal shaft 12. A first distal inflation lumen 35 for inflation of first distal occlusion element 36 and a second distal inflation lumen 49 are also positioned between proximal occlusion element 22 and proximal shaft 12. First distal guidewire 27 is positioned within first distal shaft lumen 34 and exits proximal to first distal occlusion element 36. Second distal guidewire 41 is positioned within second distal shaft lumen 48 and exits proximal to second distal occlusion element 50. First distal guidewire 27 and second distal guidewire 41 then extend proximally outside of proximal shaft 12. In this embodiment, the outer profile may be reduced to less than 6 French. Advantages of this embodiment also include ease of maneuvering with less moving parts than in the embodiments shown in FIGS. 3A-3D, for example.

In some embodiments, a single inflation lumen may be used to inflate some or all of the occlusion elements together. This design would provide a further reduced outer diameter.

Returning now to FIGS. 1A and 1B, proximal shaft 12 is positioned externally and coaxially with respect to first and second distal shafts 26 and 40, as shown in cross-section A-A, in FIG. 1B, and extends from first distal shaft proximal end 28 and second distal shaft proximal end 42 to a location proximal to first distal shaft distal end 30 and second distal shaft distal end 44. Proximal shaft lumen 20 is configured to deliver and remove a delivery substance to and from a vessel. Delivery substances may be delivered to a vessel by being introduced at hub 60 and delivered through proximal shaft lumen 20 and out through proximal shaft inlet/outlet port 24. Delivery substances may also be removed from a vessel by being introduced through proximal shaft inlet/outlet port 24 and passing though proximal shaft lumen 20 and into hub 60. A space within proximal shaft lumen 20 is determined by the outer diameters of first and second distal shafts 26 and 40. The space within proximal shaft lumen 20 must be sufficiently sized for providing the delivery substance to the vessel, and may also be sized for placement of a guidewire therethrough, such as first and/or second guidewires 27 and 41, for example.

Returning now to FIG. 1A, proximal occlusion element 22 is positioned on proximal shaft 12 at or near proximal shaft distal end 16, such that proximal occlusion element 22 is proximal to proximal shaft inlet/outlet port 24. Proximal occlusion element 22 has a proximal occlusion element proximal end 70 and a proximal occlusion element distal end 72. First distal occlusion element 36 is positioned on first distal shaft 26, at or near first distal shaft distal end 30, proximal to first distal shaft inlet/outlet port 37, and distal to proximal shaft distal end 16. First distal occlusion element 36 has a first distal occlusion element proximal end 74 and a first distal occlusion element distal end 76. Second distal occlusion element 50 is positioned on second distal shaft 40, at or near second distal shaft distal end 44, proximal to second distal shaft inlet/outlet port 51, and distal to proximal shaft distal end 16. Second distal occlusion element 50 has a second distal occlusion element proximal end 78 and a second distal occlusion element distal end 79.

In some embodiments, first distal shaft 26 may be configured to hold first distal guidewire 27 therein. In some embodiments, second distal shaft 40 may be configured to hold second distal guidewire 41 therein. Proximal shaft lumen 20 is configured to hold first and second distal shafts 26 and 40 therein and to further hold a delivery substance in between first and second distal shaft outer walls 32 and 46 and an inner wall of proximal shaft 12 within proximal shaft lumen 20. In some embodiments, proximal shaft lumen 20 is further configured to hold a guidewire therein, such as first and/or second distal guidewires 27 and 41. A delivery substance may be introduced into the vessel through proximal shaft 12 and out through proximal shaft inlet/outlet port 24, but is prevented from flowing outside of a treatment zone by inflation of proximal occlusion element 22 and inflation of first and second distal occlusion elements 36 and 50. First and second distal occlusion elements 36 and 50 are configured to be positioned in two branches of a vessel, thus allowing for a treatment area to be defined within a bifurcation.

Hub 60 is positioned at a proximal end of catheter 10 and is attached to proximal shaft 12 at proximal shaft proximal end 14. Hub 60 includes an infusion port 62 for introducing a delivery substance such as a drug solution into proximal shaft lumen 20 and a proximal occlusion element inflation port 64 for delivery of inflation fluid to proximal occlusion element 22. Hub 60 may further include a pressure monitoring port 66 and first and second distal occlusion element inflation ports and introductory ports 65, 67, 68 and 69. In some embodiments, first and/or second distal introductory ports 68 and 69 are fixed such that first and/or second distal shafts 26 and/or 40 are immovable with respect to proximal shaft 12. In other embodiments, one or both of first and second introductory ports 68 and 69 are not fixed, such that they allow for movement of first and/or second distal shafts 26 and 40 with respect to proximal shaft 12, thus enabling variability of a length of a treatment area in one or both branches.

Referring now to FIG. 1B, the configuration of proximal shaft 12 and first and second distal shafts 26 and 40 in accordance with embodiments of the present invention is shown in cross-section. Proximal shaft lumen 20 is configured to receive therein both first and second distal shafts 26 and 40, and also a delivery substance introduced via infusion port 62. Proximal shaft 12 may further include a proximal occlusion element inflation lumen 21 for introducing inflation fluid from proximal occlusion element inflation port 64 into proximal occlusion element 22. In some embodiments, proximal shaft 12 further includes a pressure lumen 92 in fluid communication with pressure monitoring port 66. Pressure lumen 92 has a proximal pressure transducer attached thereto which is capable of measuring the pressure of a column of fluid located within pressure lumen 92. Proximal shaft lumen 20 may also be configured to receive one or multiple guidewires therethrough, in between the body of proximal shaft 12 and first and second distal shaft outer walls 32 and 46. In some embodiments, first and/or second distal shafts 26 and 40 may have a first distal shaft lumen 34 and/or a second distal shaft lumen 48 for receiving a guidewire therethrough, and further may include includes first and second distal inflation lumens (shown in FIGS. 3B, 3D and 3F) for introducing inflation fluid into first and second distal occlusion elements 36 and 50. Each of first and second distal shafts 26 and 40 may further include a core wire 82 positioned therein or attached thereto. In some embodiments, core wires 82 are positioned between layers of a polymer shaft of first and/or second distal shafts 26, 40.

In embodiments of the present invention, radiopaque markers 49 may be included at or near first and second distal occlusion elements 36 and 50, proximal occlusion element 22 and other locations along catheter 10 for visualization of the position of catheter 10 within the vessel and relative positions of first and second distal and proximal occlusion elements 36, 50 and 22. In a preferred embodiment, radiopaque markers 49 are located at or just distal to proximal occlusion element distal end 72 and at or just proximal to first and second distal occlusion element proximal ends 74 and 78.

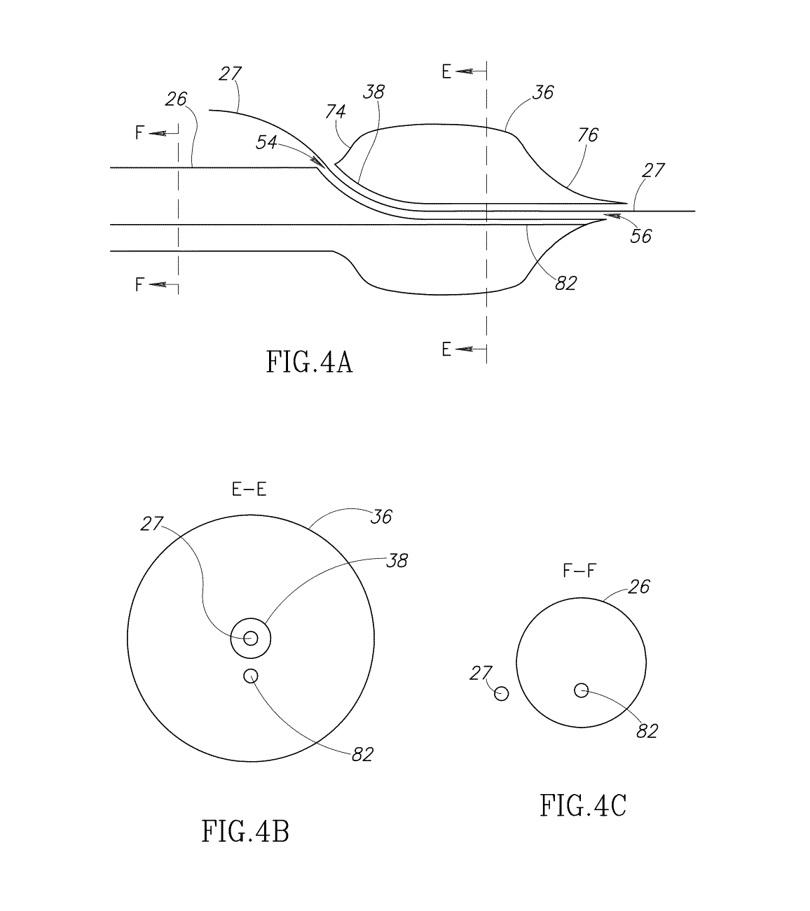

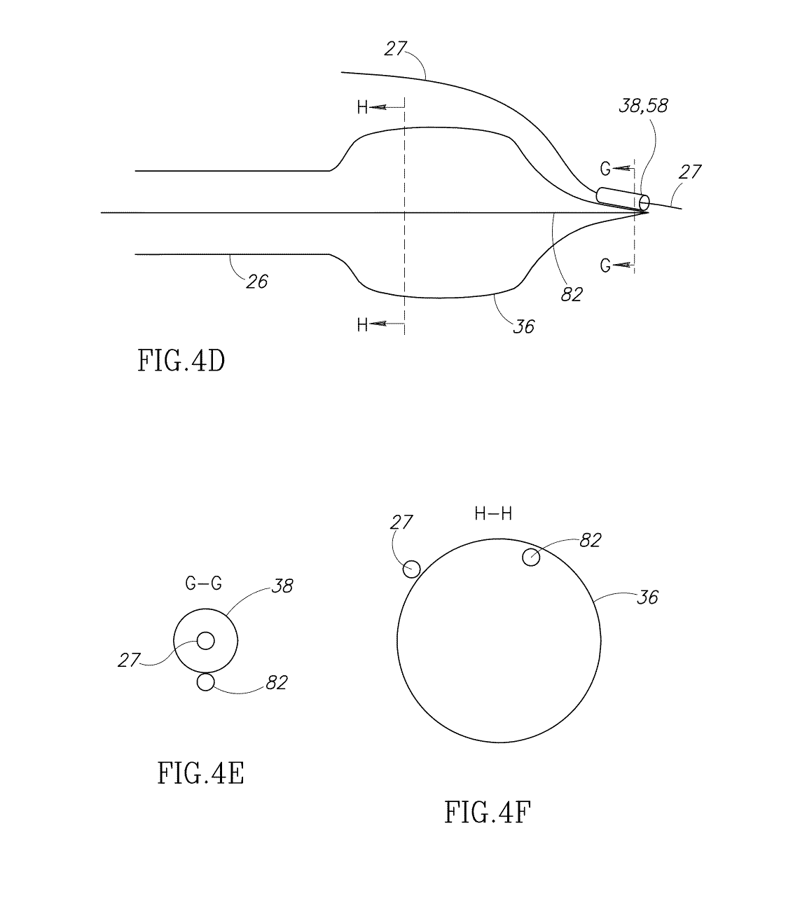

Reference is now made to FIGS. 4A-4F, which are schematic illustrations (FIGS. 4A and 4D) and cross-sectional illustrations (FIGS. 4B, 4C, 4E and 4F), respectively, of first distal shaft 26 with a first distal rail 38 in accordance with embodiments of the present invention. It should be readily apparent that second distal shaft 40 may have a second distal rail, which may include any or all of the embodiments shown herein with respect to first distal rail 38. First distal rail 38 may be useful for advancement of first distal shaft 26 into a vessel. In addition, in some embodiments, first distal rail 38 may be useful for reducing pressure that may build up in the vessel due to introduction of a delivery substance by providing controlled removal of blood from the treatment area to a location outside of the treatment area. First distal rail 38 is sized with a diameter slightly larger than a diameter of first distal guidewire 27, which is a movable guidewire positionable within first distal rail 38. For example, an inner diameter of first distal rail 38 may be approximately 0.002″ greater than a diameter of first distal guidewire 27. This difference in diameter provides a clearance space for controlled removal of blood from the treatment zone, which can be useful in preventing pressure buildup in the treatment zone when the delivery substance is introduced. In some embodiments, the clearance space is sized such that only blood can move through, and not the delivery substance, due to blood having a lower viscosity than the delivery substance. For example, contrast solution, which has a higher viscosity than blood, should not be able to move through the clearance space.

Core wire 82 may be positioned within first distal shaft 26 and may be attached to first distal occlusion element 36 at a distal end thereof and to first distal shaft 26 at first distal shaft proximal end 28 and/or at additional points along the length of first distal shaft 26.

In one embodiment, as shown in FIG. 4A, first distal rail 38 comprises a passageway through first distal occlusion element 36. In this embodiment, first distal rail 38 includes a first distal rail proximal opening 54 and a first distal rail distal opening 56, wherein first distal guidewire 27 may be introduced into catheter 10 through first distal rail distal opening 56 and may exit at first distal rail proximal opening 54 located at or near first distal occlusion element proximal end 74. Reference is made to FIG. 4B, which is a cross-sectional illustration of catheter 10 of FIG. 4A, showing a cross-section at E-E. First distal occlusion element 36 is shown, with first distal rail 38 positioned through first distal occlusion element 36 and with first distal guidewire 27 positioned within first distal rail 38. Core wire 82 is depicted as well, positioned through first distal occlusion element 36. Reference is made to FIG. 4C, which is a cross-sectional illustration of catheter 10 of FIG. 4A, showing a cross-section at F-F. At this more proximal portion of catheter 10, first distal shaft 26 has core wire 82 but does not include first distal rail 38 or first distal guidewire 27. First distal guidewire 27 is outside of first distal shaft 26 at this point.

In another embodiment, as shown in FIGS. 4D-4F, first distal rail 38 comprises a separate distal element 58 positioned on first distal shaft 26 distal to first distal occlusion element 36. In some embodiments, distal element 58 may have a length of 4-20 mm. First distal rail 38 allows for rapid exchange of catheters. First distal shaft 26 further includes core wire 82 positioned for providing stiffness through catheter 10. This enhances pushability of catheter 10. Core wire 82 is positioned within first distal shaft 26 and may be attached to first distal occlusion element 36 at a distal end thereof and to first distal shaft proximal end 28 and optionally at additional points along the length of first distal shaft 26. In some embodiments, core wire 82 is sandwiched between polymeric layers of first distal shaft 26. Reference is made to FIG. 4E, which is a cross-sectional illustration of catheter 10 of FIG. 4D, showing a cross-section at G-G. Distal rail 38 is shown, with first distal guidewire 27 positioned therethrough. Core wire 82 is depicted as well, positioned outside of first distal rail 38. Reference is made to FIG. 4F, which is a cross-sectional illustration of catheter 10 of FIG. 4D, showing a cross-section at H-H. At this more proximal portion of catheter 10, first distal occlusion element 36 is shown with core wire 82 positioned therethrough. First distal guidewire 27 is outside of first distal shaft 26 at this point.

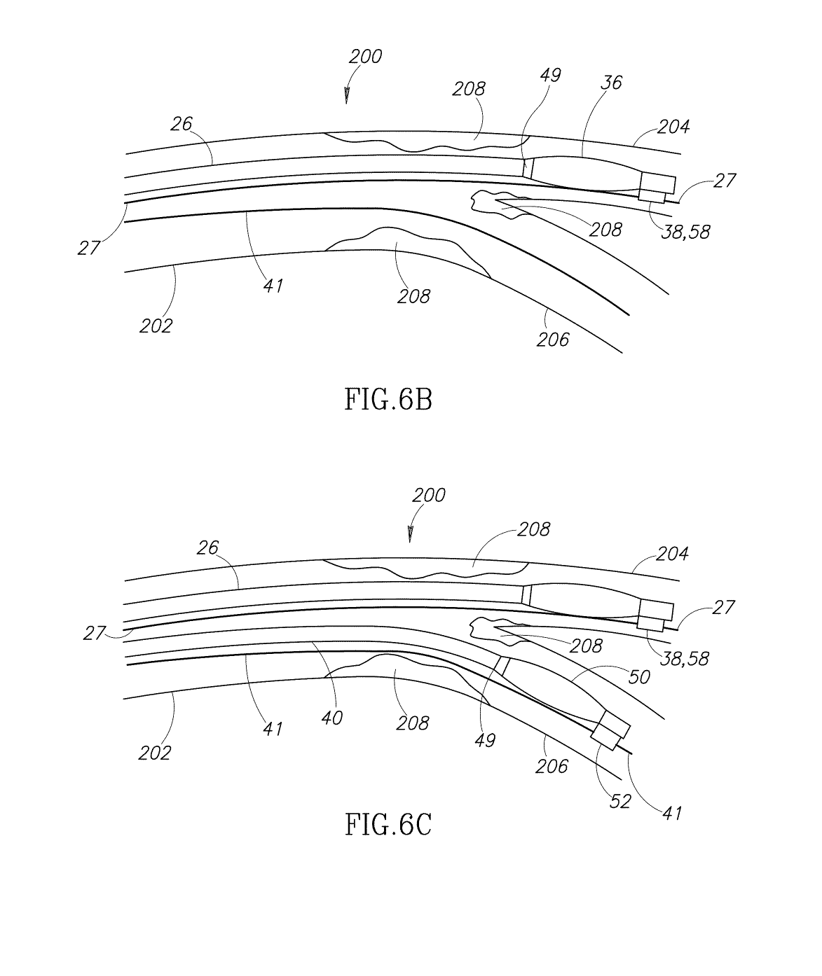

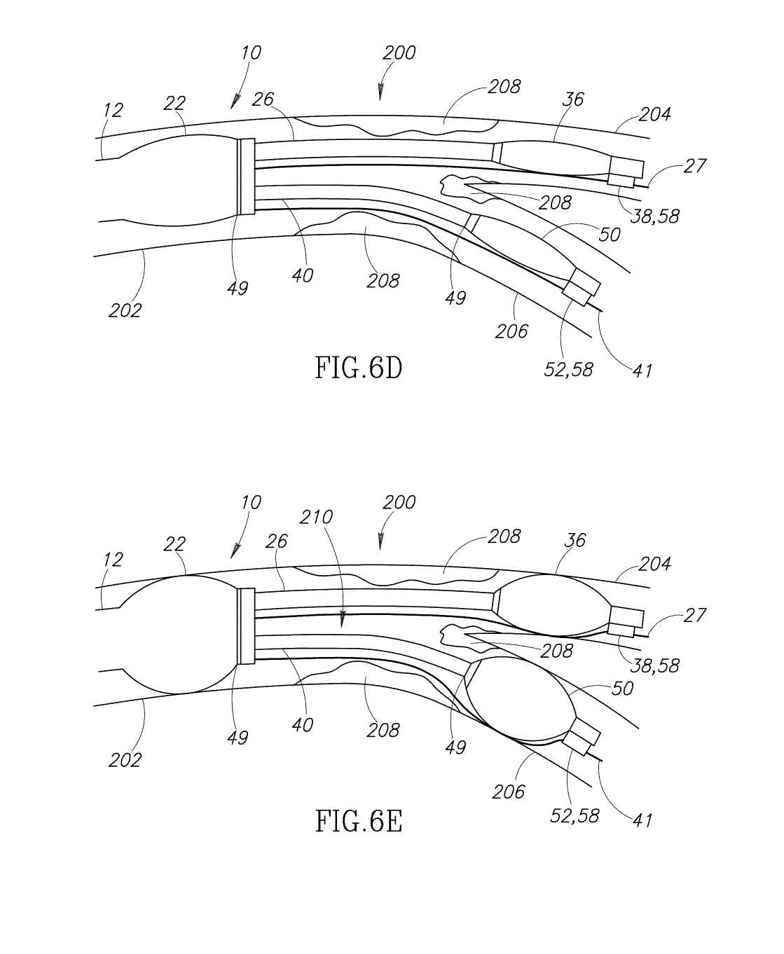

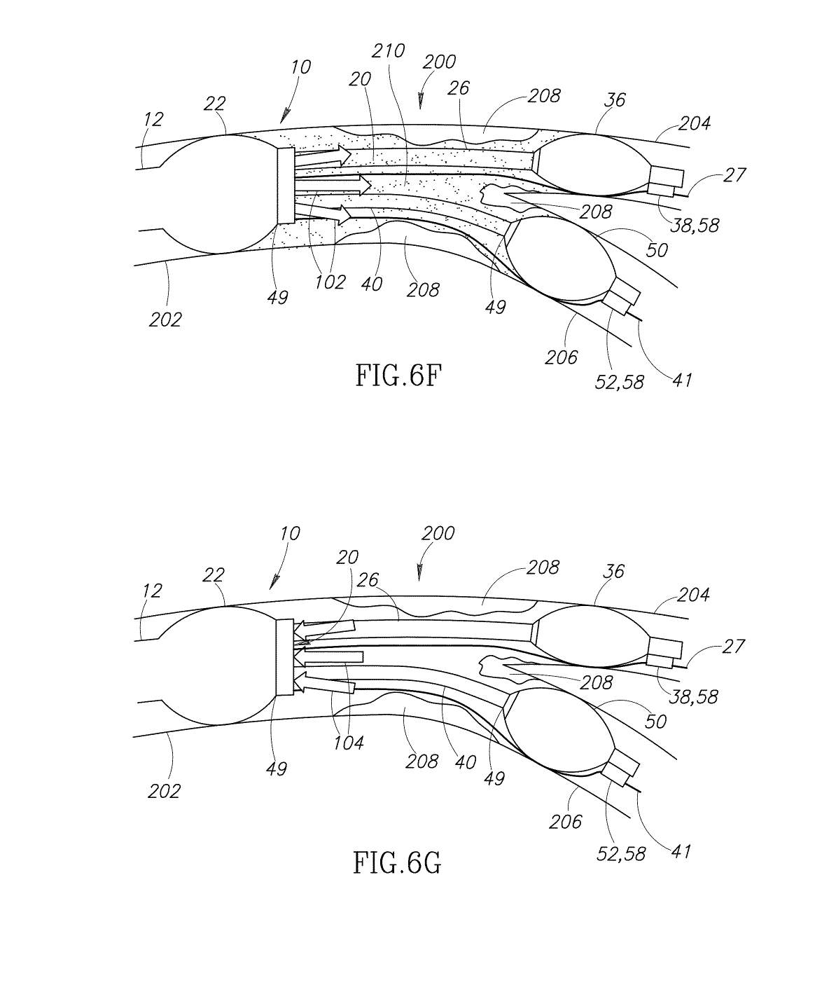

First distal rail 38 may provide added advantages because it provides an additional lumen within catheter 10. For the embodiments shown in FIGS. 4A-4F wherein first distal rail 38 may be used with first distal guidewire 27, proximal shaft lumen 20 may house first distal guidewire 27 when first distal shaft 26 and second distal shaft 40 are positioned within proximal shaft 12. In these embodiments, first distal shaft 26 and second distal shaft 40 may be initially positioned within proximal shaft 12, and first distal guidewire 27 is introduced through distal rail 38 of first distal shaft 26. Catheter 10, including all three of first distal shaft 26, second distal shaft 40 and proximal shaft 12, is advanced over first distal guidewire 27. Once first distal guidewire 27 is positioned within first distal rail 38, first distal guidewire 27 is further positioned within proximal shaft lumen 20. This positioning allows for an over the wire type of advancement, but with a reduced profile, since an additional over the wire lumen is not required. In this case, one or both of first distal shaft lumen 34 and second distal shaft lumen 48 may be eliminated thus reducing the profile of catheter 10. In some embodiments, a second distal rail 52 (shown in FIG. 1A) is included in second distal shaft 40, and second distal guidewire 41 may be used as well, as will be described and shown hereinbelow with reference to FIGS. 6A-6G.

In some embodiments, one or both of first distal shaft lumen 34 and second distal shaft lumen 48 may be maintained and used for other items even when not in use for first and/or second distal guidewire 27, 41. For example, a mandrel may be introduced through first and/or second distal shaft lumen 34, 48 for enhancing pushability and for advancing first and/or second distal shaft 26, 40. In some embodiments, either or both of first/second distal shaft lumen 34, 48 may be used for exchanging guidewires, or for putting a second or third guidewire in the vessel. Alternatively, either or both of first/second distal shaft lumen 34, 48 may be used for perfusion. For example, in a case of prolonged occlusion while treating the vessel, blood may be introduced through either or both of first/second distal shaft lumen 34, 48 to an area distal to first and/or second distal occlusion element 36, 50, thus making it possible to keep treating the vessel for as long as necessary. This may be particularly useful in the coronary arteries, for example, which cannot be occluded for a prolonged period of time. In some embodiments, blood may be cooled or otherwise treated and then introduced through either or both of first/second distal shaft lumen 34, 48. In some embodiments, a supply shaft having a supply lumen, such as for example, a vascular sheath, is introduced coaxial to catheter 10 for removing blood from the vessel. This blood may then be reintroduced through either or both of first/second distal shaft lumen 34, 48. In some embodiments, either or both of first/second distal shaft 26, 40 may be removed from proximal shaft 12 during a procedure.

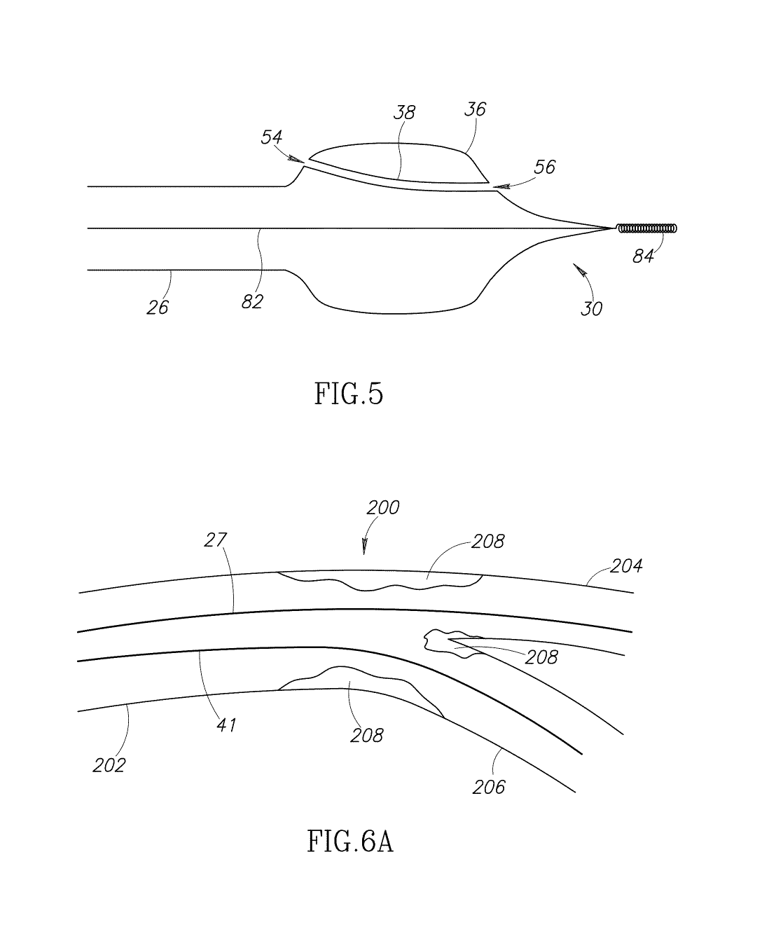

Reference is now made to FIG. 5, which is an illustration of first distal shaft 26 in accordance with yet another embodiment. In this embodiment, distal rail 38 may be a passageway, as depicted in FIG. 4A. However, distal rail 38 has a smaller diameter and may not be used for placement of a movable guidewire therethrough. For example, first distal rail 38 may have a diameter of 0.001″-0.002″—enough for blood to flow through, but not enough for a delivery substance due to its higher viscosity, and not enough for a movable guidewire. In this embodiment, a fixed wire 84 may be positioned at first distal shaft distal end 30. Thus, for example, first distal occlusion element 36 may be a fixed wire balloon. In some embodiments, an additional movable wire may be introduced through second distal shaft lumen 48 and/or through proximal shaft lumen 20. In yet another embodiment, first distal rail 38 is sized for a movable guidewire and in addition a fixed wire 84 is used as well.

Proximal and first and second distal occlusion elements 22, 36, 50 are comprised of an atraumatic surface so as not to damage the inner walls of a blood vessel. In a preferred embodiment, proximal and first and second distal occlusion elements 22, 36, 50 have a hydrophilic surface, which by attracting water forms a natural atraumatic layer. Furthermore, a hydrophilic surface can provide means for occlusion which is configured to open when in contact with water components from the blood. Proximal and first and second distal occlusion elements 22, 36, 50 may further include a coating for providing long-term (measured in hours, days or even months) implantation of catheter 10 in the body. Alternatively or in addition, proximal and first and second distal occlusion elements 22, 36, 50 may further include a drug coating. In one embodiment, proximal and first and second distal occlusion elements 22, 36, 50 are balloons, such as are commonly used with catheter systems, and are expandable by introduction of a fluid therein, wherein the fluid can be a liquid or a gas. In this embodiment, separate inflation lumens are included within catheter 10, either alongside or coaxial with first or second distal shafts 26, 40, and are in fluid communication with proximal and first and second distal occlusion elements 22, 36, 50. Fluid is introduced via inflation ports 64, 65, 67 positioned at hub 60. These types of balloons and inflation lumens are commonly known in the art. The balloon may be elastomeric, compliant, semi-compliant or non-compliant, as long as it serves to occlude the vessel without causing damage to the internal walls. In one embodiment, the balloon is pre-formed and relatively thin, so as to reduce the pressure necessary to inflate the balloon, while keeping the outer diameter to a minimum. For example, balloon thickness may range from 0.0001 inches to 0.002 inches, a range which is smaller than thicknesses of standard occlusion balloons.

In another embodiment, proximal and first and second distal occlusion elements 22, 36, 50 are self-expanding elements confined within retractable sheaths, such that upon retraction of the sheath, the self-expanding element expands to a diameter sufficient to occlude the vessel. In this embodiment, the sheath is connected to a retractor positioned at a proximal end of catheter 10. The self-expanding element may be comprised of an elastic or spring-like material, or a shape-memory alloy. Such materials are known in the art. In another embodiment, proximal and first and second distal occlusion elements 22, 36, 50 are comprised of a mechanically actuated mechanism, whereby they are expanded by mechanical means. In yet another embodiment, proximal and first and second distal occlusion elements 22, 36, 50 are comprised of a temperature sensitive material which can be expanded or retracted by exposure to specific temperatures. Specifically, perfusion of cooled or heated blood through catheter 10 would cause expansion of proximal and first and second distal occlusion elements 22, 36, 50, and perfusion of normothermic blood through catheter 10 (such as, for example, during renormalization of temperature) would cause retraction of proximal and first and second distal occlusion elements 22, 36, 50. This may be accomplished, for example, by using a shape-memory material, either as proximal and first and second distal occlusion elements 22, 36, 50 themselves, or as an actuator positioned alongside proximal and first and second distal occlusion elements 22, 36, 50. Similarly, this could be accomplished by using a bi-metallic strip. In one embodiment, proximal and first and second distal occlusion elements 22, 36, 50 are an integral part of the catheter, wherein a portion of catheter 10 having a slightly wider diameter is configured to be wedged into the vessel, and thus acts as occlusion element, providing both occlusion and anchoring functionality.

Proximal and first and second distal occlusion elements 22, 36, 50 further include radiopaque markers 49 for viewing of a location of catheter 10 generally and a location of proximal and first and second distal occlusion elements 22, 36, 50 specifically within the vessel. In one embodiment, proximal and first and second distal occlusion elements 22, 36, 50 are themselves comprised of radiopaque material. In alternative embodiments, one or more radiopaque markers 49 are positioned on or adjacent to proximal and first and second distal occlusion elements 22, 36, 50. Additional radiopaque markers 49 may also be positioned in other places along catheter 10 such as, for example, at proximal shaft distal end 16. In one embodiment, a radiopaque marker 49 is positioned at the distal tip of catheter 10. Radiopaque marker 49 can be a ring surrounding the distal tip, or, in order to minimize stiffness at the tip, a radiopaque marker may be comprised of a small sliver of radiopaque material embedded within a portion of the distal tip. In one embodiment, radiopaque marker 49 is filled with an adhesive and positioned so as to seal an inflation lumen for inflation of proximal and first and second distal occlusion elements 22, 36, 50.

In some embodiments, first and/or second distal shafts 26 and 40 are introduced into the bifurcated branches of a vessel, and proximal shaft 12 is introduced over first and second distal shafts 26, 40. In other embodiments, proximal shaft 12 is advanced into a vessel first with a guidewire positioned through proximal shaft lumen 20, followed by advancement of first and second distal shafts 26 and 40. First and second distal shafts 26 and 40 may be advanced through proximal shaft lumen 20, resulting in the guidewire and first and second distal shafts 26 and 40 all positioned alongside one another within proximal shaft lumen 20. In other embodiments, proximal shaft 12 and first distal shaft 26 are advanced together into the vessel. In one embodiment, this may be done as an over the wire system, wherein a guidewire is introduced into the vessel and then positioned within first distal shaft lumen 34, whereupon catheter 10 is advanced over the guidewire, or may be done using first distal rail 38. Second distal shaft 40 may then be advanced through proximal shaft lumen 20. In yet another embodiment, all three of proximal shaft 12, first distal shaft 26 and second distal shaft 40 are advanced together into the vessel. This may be done as an over the wire system, or it may be done using first and second distal rail 38, 52, or it may be done as a partially over the wire system and partially using a distal rail.

Reference is now made to FIGS. 6A-6G, which are schematic illustrations showing a method of using catheter 10 in accordance with embodiments of the present invention.

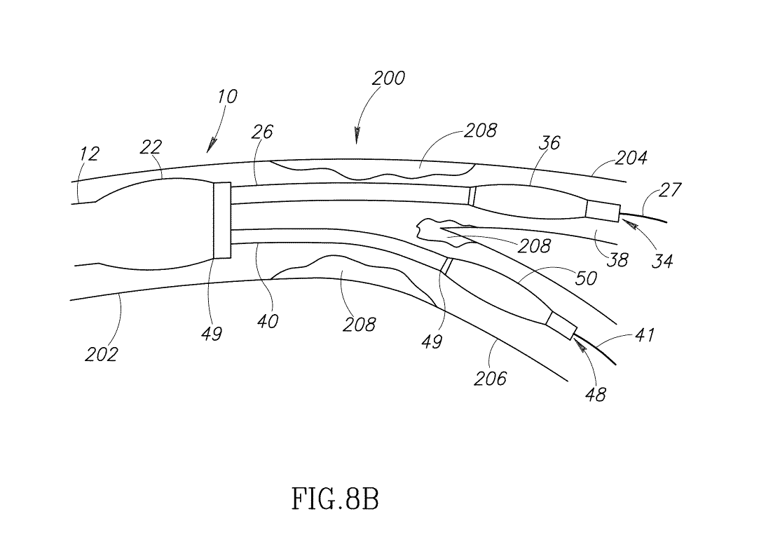

A bifurcated vessel 200 is shown having a proximal branch 202, a first distal branch 204 and a second distal branch 206. Bifurcated vessel 200 is shown with a lesion 208 at the area of bifurcation. As shown in FIG. 6A, first distal guidewire 27 is introduced into vessel 200 and distally into first distal branch 204. Second distal guidewire 41 is introduced into vessel 200 and distally into second distal branch 206. As shown in FIG. 6B, first distal shaft 26 is introduced over first distal guidewire 27 and advanced into first distal branch 204 until first distal shaft 26 is positioned past lesion 208, as determined by radiopaque marker 49. First distal shaft 26 may be advanced via first distal rail 38 as shown in FIG. 6B, or may be advanced over the wire via first distal shaft lumen 34 (shown with reference to another embodiment in FIG. 8B). Although first distal rail 38 is shown in the drawings as distal element 58 of FIG. 4D, first distal rail 38 in some embodiments is a passageway with first distal rail proximal opening 54 and a first distal rail distal opening 56 as in FIG. 4A and FIG. 5. As shown in FIG. 6C, second distal shaft 40 is introduced over second distal guidewire 41 and advanced into second distal branch 206 until second distal shaft 40 is positioned past lesion 208, as determined by radiopaque marker 49. Second distal shaft 40 may be advanced via second distal rail 52 as shown in FIG. 6C, or may be advanced over the wire via second distal shaft lumen 48 (shown with reference to another embodiment in FIG. 8B). Although second distal rail 52 is shown in the drawings as distal element 58 of FIG. 4D, second distal rail 52 in some embodiments is a passageway with first distal rail proximal opening 54 and a first distal rail distal opening 56 as in FIG. 4A and FIG. 5. Next, as shown in FIG. 6D, proximal shaft 12 is advanced over first and second distal shafts 26 and 40 into proximal branch 202 until proximal occlusion element 22 is proximal to lesion 208, as determined by marker 49. In some embodiments, proximal shaft 12, and first and second distal shafts 26 and 40 are first assembled together outside of the body and then advanced together into vessel 200 over first and second distal guidewires 27, 41. In some embodiments, proximal shaft 12 is advanced over an additional movable guidewire, and may be advanced prior to first and second distal shafts 26 and 40 or at the same time. Next, as shown in FIG. 6E, proximal occlusion element 22, first distal occlusion element 36 and second distal occlusion element 50 are inflated, thus defining a treatment zone 210 within vessel 200. Inflation of proximal occlusion element 22, first distal occlusion element 36 and second distal occlusion element 50 may be done simultaneously or sequentially. Since each of proximal occlusion element 22, first distal occlusion element 36 and second distal occlusion element 50 is separately inflatable, each or multiple of proximal occlusion element 22, first distal occlusion element 36 and second distal occlusion element 50 may be inflated and deflated as necessary. In some embodiments, inflation lumens may be combined, either for two of the three occlusion elements or for all three together in order to reduce the overall profile. In these embodiments, inflation would necessarily be done at the same time for those occlusion elements with a shared inflation lumen. Next, as shown in FIG. 6F, a delivery substance such as a drug solution is introduced into treatment zone 210 through proximal shaft lumen 20, as depicted by outgoing arrows 102. Since the proximal and distal borders are defined by proximal occlusion element 22, first distal occlusion element 36 and second distal occlusion element 50, the delivery substance remains within treatment zone 210 for a period of time, which can last as long as proximal occlusion element 22, first distal occlusion element 36 and second distal occlusion element 50 remain inflated. As shown in FIG. 6G, the delivery substance may be fully or partially removed from treatment zone 210 via proximal shaft lumen 20, as depicted by incoming arrows 104. Proximal occlusion element 22, first distal occlusion element 36 and second distal occlusion element 50 are then deflated, and catheter 10 is removed from vessel 200.

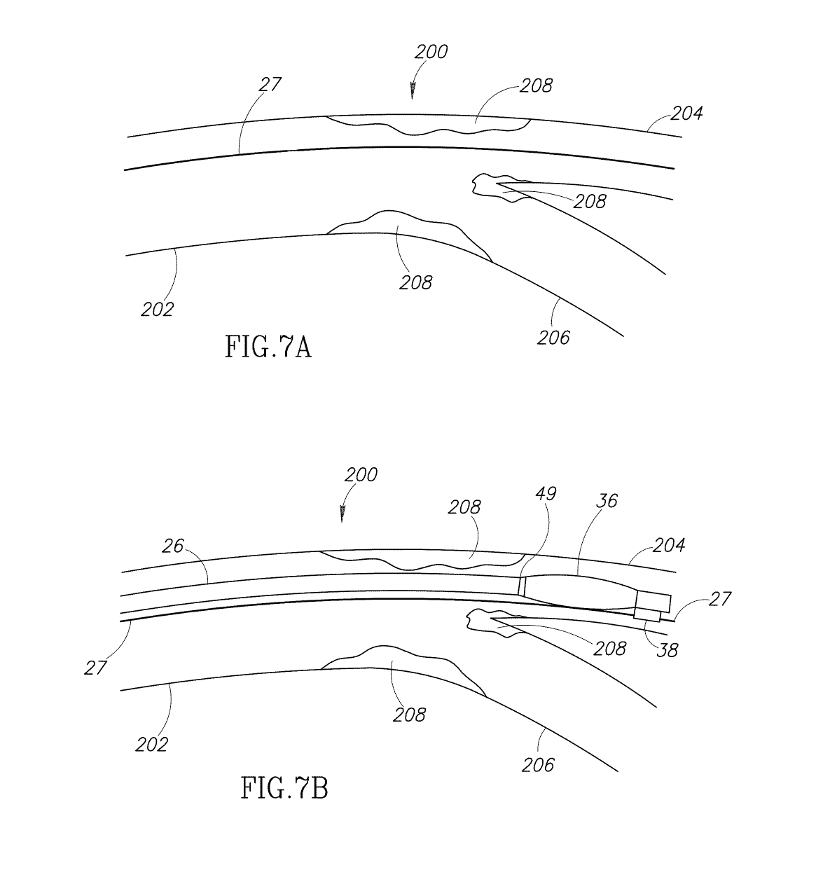

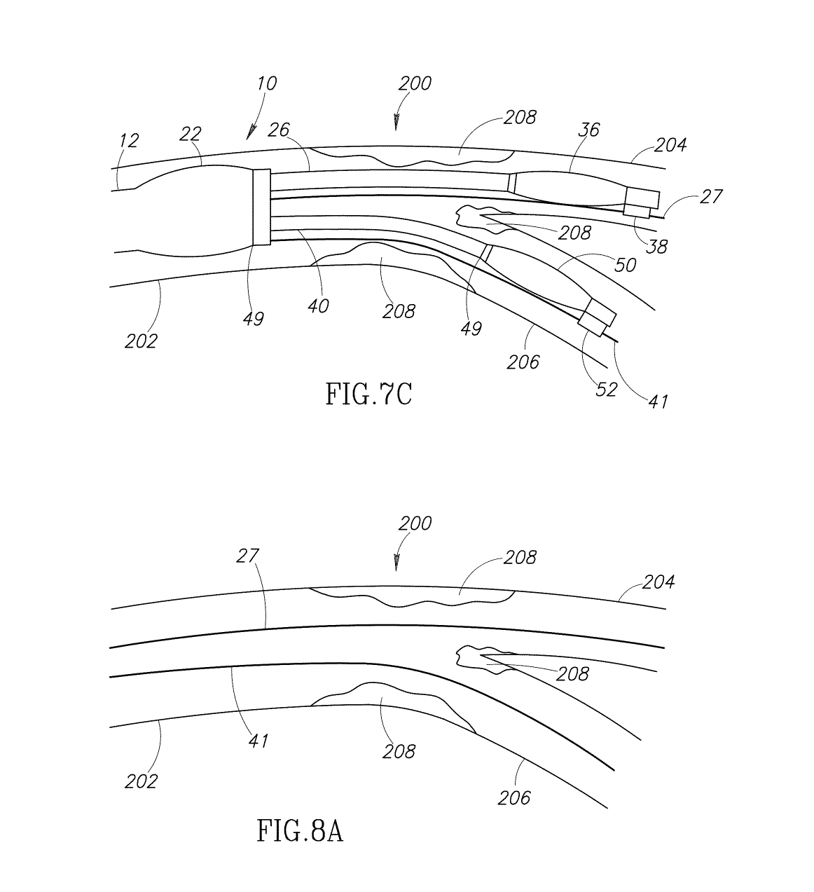

Reference is now made to FIGS. 7A-7C, which are schematic illustrations showing a method of using catheter 10 in accordance with additional embodiments of the present invention. In this embodiment, second distal shaft 40 is attached to proximal shaft 12. As shown in FIG. 7A, first distal guidewire 27 is introduced into vessel 200 and into first distal branch 204. As shown in FIG. 7B, first distal shaft 26 is then advanced over first distal guidewire 27 and advanced into first distal branch 204 until first distal shaft 26 is positioned past lesion 208, as determined by radiopaque marker 49. First distal shaft 26 may be advanced via first distal rail 38 as shown in FIG. 7B, or may be advanced over the wire via first distal shaft lumen 34 (shown with reference to another embodiment in FIG. 8B). Next, as shown in FIG. 7C, proximal shaft 12 with second distal shaft 40 attached thereto is advanced over first distal shaft 26 into proximal branch 202 until proximal occlusion element 22 is proximal to lesion 208 and second distal shaft 40 is distal to lesion 208, as determined by markers 49. The method may then continue in accordance with steps described above with reference to FIGS. 6E-6G.

Reference is now made to FIGS. 8A and 8B, which are schematic illustrations showing a method of using catheter 10 in accordance with additional embodiments of the present invention. In this embodiment, first and second distal shafts 26 and 40 are attached to proximal shaft 12. First, as shown in FIG. 8A, first distal guidewire 27 is introduced into vessel 200 and distally into first distal branch 204. In some embodiments, second distal guidewire 41 is introduced into vessel 200 and distally into second distal branch 206. In other embodiments, only first distal guidewire 27 is used. Next, as shown in FIG. 8B, proximal shaft 12 with first and second distal shafts 26 and 40 attached thereto is advanced together into vessel 200 over first and second distal guidewires 27 and 41. Advancement may be done over the wire, as shown in FIG. 8B, wherein first distal guidewire 27 is positioned through first distal shaft lumen 34 and second distal guidewire 41 is positioned through second distal shaft lumen 48, or it may be done via first and second distal rails 38 and 52, as described above with reference to FIGS. 6B and 6C. In yet additional embodiments, one of first and second distal shafts 26 and 40 may be advanced over the wire and the other one advanced via a distal rail. In yet additional embodiments, proximal shaft 12 may be advanced over a guidewire, and first and second distal shafts 26 and 40 may have fixed wire balloons. The method may then continue in accordance with steps described above with reference to FIGS. 6E-6G.

### Examples

### Experiment 1

An experiment was performed using a porcine model, wherein the catheter of FIG. 1A was deployed on the right side via the right superficial femoral artery. A first distal balloon occluded the right iliac artery and a second distal balloon occluded the right deep femoral artery. A proximal balloon was positioned in the right superficial femoral artery. The three balloons were inflated, and a contrast agent was held within the area defined by the three balloons. The contrast was held with the area for a period of up to 6 minutes, with angiographic images taken at several points during the procedure.

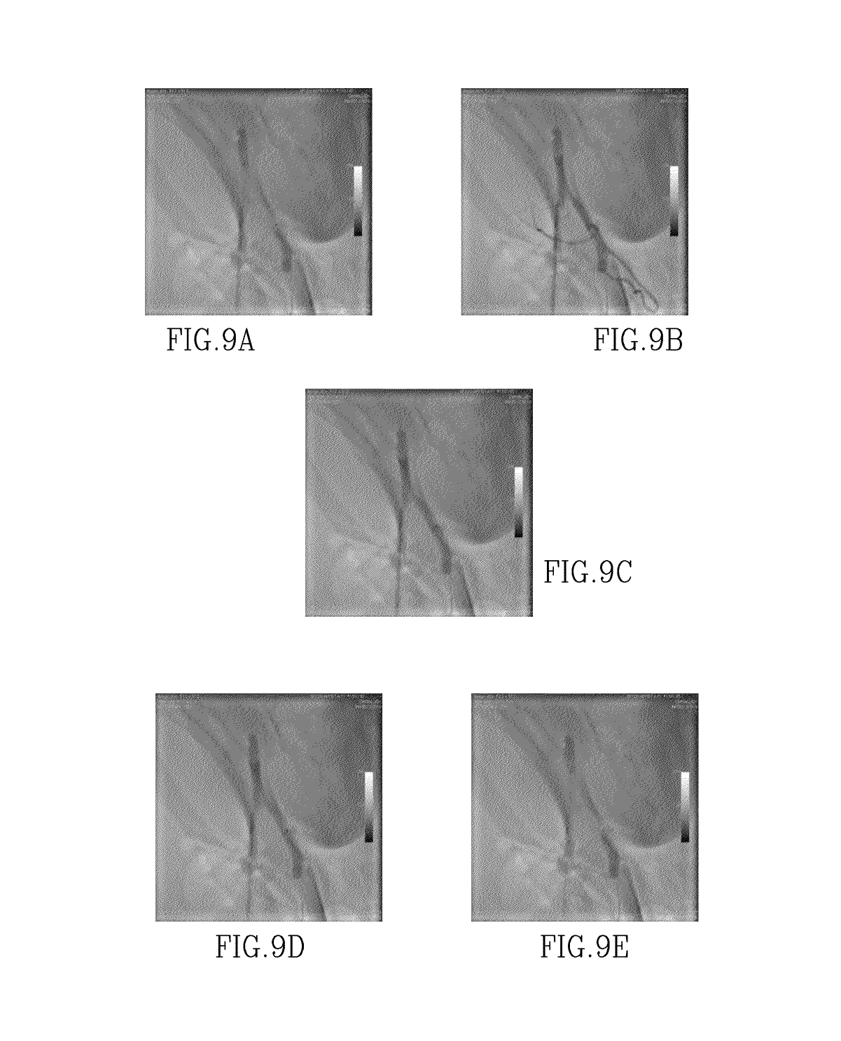

Reference is made to FIGS. 9A-9E, which are angiographic images taken during the procedure at a time T=0 (FIG. 9A), T=0 minutes, 5 seconds (FIG. 9B), T=3 minutes (FIG. 9C), T=6 minutes (FIG. 9D) and T=6 minutes, 4 seconds (FIG. 9E) at which point the contrast agent was removed from the vessel. It is apparent from these angiographic images that a catheter such as the one described above can be useful in treating a bifurcated vessel for extended periods of time.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A catheter for delivery of agents to a bifurcated vessel, the catheter comprising:
a proximal shaft having
a proximal shaft proximal end,
a proximal shaft distal end,
a proximal shaft outer wall extending from said proximal shaft proximal end to said proximal shaft distal end, said proximal shaft outer wall defining a proximal shaft lumen, and
a proximal occlusion element positioned at said proximal shaft distal end;
a first distal shaft positioned within said proximal shaft lumen, said first distal shaft having
a first distal shaft proximal end,
a first distal shaft distal end,
a first distal shaft outer wall extending from said first distal shaft proximal end to said first distal shaft distal end, and
a first distal occlusion element positioned at said first distal shaft distal end; and
a second distal shaft positioned within said proximal shaft lumen alongside said first distal shaft, said second distal shaft having
a second distal shaft proximal end,
a second distal shaft distal end,
a second distal shaft outer wall extending from said second distal shaft proximal end to said second distal shaft distal end, and
a second distal occlusion element positioned at said second distal shaft distal end.

2. The catheter of claim 1 further comprising a first distal rail at said first distal shaft distal end.

3. The catheter of claim 2 further comprising a second distal rail at said second distal shaft distal end.

4. The catheter of claim 1, wherein said first distal shaft outer wall defines a first distal shaft lumen.

5. The catheter of claim 1, wherein said second distal shaft outer wall defines a second distal shaft lumen.

6. The catheter of claim 1, wherein said first distal shaft is slidingly movable with respect to said proximal shaft and wherein said first distal occlusion element is at a variable distance from said proximal occlusion element.

7. The catheter of claim 6, wherein said second distal shaft is slidingly movable with respect to said proximal shaft and wherein said second distal occlusion element is at a variable distance from said proximal occlusion element.

8. The catheter of claim 1, wherein said first distal shaft is attached to said proximal shaft, and wherein said first distal occlusion element is at a fixed distance from said proximal occlusion element.

9. The catheter of claim 8, wherein said second distal shaft is attached to said proximal shaft, and wherein said second distal occlusion element is at a fixed distance from said proximal occlusion element.

10. The catheter of claim 1, further comprising a radiopaque marker at a proximal occlusion element distal end.

11. The catheter of claim 1, further comprising a radiopaque marker at a first distal occlusion element proximal end.

12. The catheter of claim 11, further comprising a radiopaque marker at a second distal occlusion element proximal end.

13. The catheter of claim 1, further comprising an inflation lumen positioned through said proximal shaft and configured to inflate said proximal occlusion element, said first distal occlusion element and said second distal occlusion element.

14. A method of treating a bifurcated vessel, the method comprising:
providing a catheter having a proximal shaft having a proximal occlusion element at a distal end thereof; a first distal shaft positioned within said proximal shaft, said first distal shaft having a first distal occlusion element at a distal end thereof; and a second distal shaft positioned within said proximal shaft, said second distal shaft having a second distal occlusion element at a distal end thereof;
placing a first movable guidewire into a first branch vessel;
placing a second movable guidewire into a second branch vessel;
positioning said first distal shaft on said first movable guidewire and said second distal shaft on said second movable guidewire;
advancing said catheter into the bifurcated vessel via said first and second movable guidewires;
positioning said proximal shaft in a proximal portion of the bifurcated vessel;
positioning said first distal shaft in a first distal branch portion of the bifurcated vessel;
positioning said second distal shaft in a second distal branch portion of the bifurcated vessel;
after said positioning of said proximal shaft, said first distal shaft and said second distal shaft, deploying said proximal occlusion element, said first distal occlusion element, and said second distal occlusion element; and
introducing a delivery substance through said proximal shaft.

15. The method of claim 14, wherein said advancing said catheter is done by advancing said proximal shaft, said first distal shaft and said second distal shaft simultaneously.

16. The method of claim 14, wherein said advancing said catheter is done by first advancing said first distal shaft and said second distal shaft, and then advancing said proximal shaft over said first and second distal shafts.

17. The method of claim 14, wherein said advancing said catheter is done by placing said first movable guidewire through a first distal rail positioned on a distal end of said first distal shaft.

18. The method of claim 17, wherein said advancing said catheter is done by also placing said second movable guidewire through a second distal rail positioned on a distal end of said second distal shaft.

19. The method of claim 17, wherein said advancing said catheter is done by placing said first movable guidewire through a first distal shaft lumen.

20. The method of claim 19, wherein said advancing said catheter is done by also placing said second movable guidewire through a second distal shaft lumen.

21. The method of claim 14, further comprising: after a period of time following said introducing a delivery substance, removing the delivery substance from the vessel through said proximal shaft; undeploying said proximal occlusion element, said first distal occlusion element, and said second distal occlusion element; and removing said catheter from the vessel.
